(19) United States
(12) Patent Application Publication
(10) Pub. No.: US 2022/0405957 A1
(43) Pub. Date: Dec. 22, 2022

(54) Computer Vision Systems and Methods for Time-Aware Needle Tip Localization in 2D Ultrasound Images

(71) Applicant: Rutgers, The State University of New Jersey, New Brunswick, NJ (US)

(72) Inventors: Cosmas Mwikirize, Kawoko (UG); John L. Nosher, Basking Ridge, NJ (US); Ilker Hacihaliloglu, Jersey City, NJ (US)

(73) Assignee: Rutgers, The State University of New Jersey, New Brunswick, NJ (US)

(21) Appl. No.: 17/842,207

(22) Filed: Jun. 16, 2022

Related U.S. Application Data
(60) Provisional application No. 63/212,330, filed on Jun. 18, 2021.

(51) Int. Cl. G06T 7/70; G06N 3/04; A61B 34/20

(52) U.S. Cl. CPC G06T 7/70; G06N 3/0445; A61B 34/20; G06T 2207/10016; G06T 2207/20221; G06T 2207/20084; G06T 2207/10132; A61B 2034/2063

(57) ABSTRACT

Computer vision systems and methods for time-aware needle tip localization in two-dimensional (2D) ultrasound images are provided. A consecutive fused image sequence, derived from fusion of the enhanced frames and the corresponding B-mode frames, is processed by a time-aware neural network which includes a unified convolutional neural network (CNN) and a long short-term memory (LSTM) recurrent neural network. The CNN acts as a feature extractor, with stacked convolutional layers which progressively create a hierarchy of more abstract features. The LSTM models temporal dependencies in time-series data. The system learns spatiotemporal features associated with needle tip movement, for example, needle tip appearance and trajectory information, and successfully localizes the needle tip in the presence of abrupt intensity changes and motion artifacts.

### RELATED APPLICATIONS

The present application claims the benefit of U.S. Provisional Application Ser. No. 63/212,330 filed on Jun. 18, 2021, the entire disclosure of which is expressly incorporated herein by reference.

### BACKGROUND

### Technical Field

The present disclosure relates to computer vision systems and methods. More specifically, the present disclosure relates to computer vision systems and methods for time-aware needle tip localization in two-dimensional (2D) ultrasound images.

### RELATED ART

Needle tip localization during minimally invasive ultrasound-guided procedures such as regional anesthesia and biopsies is of significant interest in the medical imaging community. These procedures are typically performed using conventional 2D B-mode ultrasound, and the needle may be inserted using one of two techniques: in-plane, where the needle is inserted parallel to the ultrasound beam, or out-of-plane, where the needle insertion plane and the ultrasound beam are perpendicular. Although in-plane insertion should ideally produce a conspicuous needle shaft and tip, it is common for the needle to veer away from the narrow field of view, producing no shaft and/or a low-intensity tip. Out-of-plane insertion, on the other hand, usually produces no shaft information and a low-intensity tip.

In either case, the interventional radiologist must rely on recognizing low-intensity features associated with tip motion while concurrently manipulating the ultrasound transducer and the needle, a challenging task which is exacerbated by motion artifacts, noise, and high-intensity anatomical artifacts. Therefore, accurate and consistent visualization of the needle tip is often difficult to achieve. Consequently, it is common for a non-experienced radiologist to miss the anatomical targets, and this could lead to injury, increased hospital stays, and reduced efficacy of procedures.

To address this challenge, several methods have been proposed, and these can broadly be categorized as hardware or software based. On the hardware front, mechanical needle guides, which are designed to keep the needle aligned with the ultrasound beam, are prominent. Some needle guides have predetermined angles of approach, while others permit minor adjustments, but overall, needle guides are inefficient in procedures where fine trajectory adjustments are required, or out-of-plane insertion is desired. Another method involves the integration of sensors at the needle tip, but this makes the needles more expensive. Three- and/or four-dimensional (3D/4D) ultrasound gives a wider field of view, overcoming the limitations of 2D ultrasound, but current technology has poor resolution and a low frame rate, making it unsuitable for real-time applications. Electromagnetic/optical tracking systems have been proposed, but they necessitate specialized needles and probes, thus adding a huge cost to the basic ultrasound system. Furthermore, electromagnetic systems are susceptible to interference from metallic objects in the operating environment. Lastly, robotic systems facilitate autonomous or semi-autonomous needle insertion, but they are expensive and not practical for routine procedures.

Software-based methods, on the other hand, rely on image analysis methods applied to the B-mode ultrasound images, to facilitate automatic needle recognition. One approach involves a method for needle localization, utilizing an Adaboost classifier and beam-steered ultra-sound images. This approach requires a visible needle shaft, which is easier to obtain on ultrasound systems with beam steering capability and difficult otherwise. Moreover, this method would not work for out-of-plane needles. Another approach presents a learning-based method for segmentation of imperceptible needle motion, relying on optical flow and support vector machines, but the method is computationally expensive (1.18 s per frame). Still other approaches have proposed a framework for needle detection in 3D ultrasound, using orthogonal-plane convolutional networks. As earlier noted, 3D ultrasound is not widely available, and 2D ultrasound remains the standard of care.

Deep learning approaches for needle shaft and tip localization have also been proposed, based on convolutional neural networks (CNNs). One instance focuses on needle tip localization for in-plane needles, in individual frames, when the shaft is at least partially visible. Other methods have been proposed targeting challenging procedures where the needle shaft may not be visible. This latter work employed a novel foreground detection scheme, in which the needle tip feature is extracted from consecutive frames, using dynamic background information. The enhanced needle frames are then fed to CNNs, one at a time, for needle tip localization. Although the methods achieved good tip localization accuracy and high computational efficiency, tip localization was affected by motion artifacts in the clinical setting, for example, those arising from physiological activity such as breathing or pulsation.

What would be desirable is a more robust and accurate needle tip localization strategy, suitable for localization of both in-plane and out-of-plane needles under 2D ultrasound guidance, in which there is no shaft information. Accordingly, the computer vision systems and methods of the present disclosure address the foregoing and other needs.

### SUMMARY

Computer vision systems and methods for time-aware needle tip localization in two-dimensional (2D) ultrasound images are provided. A consecutive fused image sequence, derived from fusion of the enhanced frames and the corresponding B-mode frames, is processed by a time-aware neural network which includes a unified convolutional neural network (CNN) and a long short-term memory (LSTM) recurrent neural network. The CNN acts as a feature extractor, with stacked convolutional layers which progressively create a hierarchy of more abstract features. The LSTM models temporal dependencies in time-series data. The combination of CNNs and LSTMs is thus able to capture time dependencies on features extracted by convolutional operations, thus supporting sequence prediction. The system learns spatiotemporal features associated with needle tip movement, for example, needle tip appearance and trajectory information, and successfully localizes the needle tip in the presence of abrupt intensity changes and motion artifacts. The systems provides a novel CNN-LSTM learning approach, optimized for learning temporal relationships emanating from needle tip motion events. Since the system does not rely on needle shaft visibility, it is appropriate for the localization of thin needles and both in-plane and out-of-plane trajectories. The systems and methods can be integrated a computer-assisted interventional system for needle tip localization.

### BRIEF DESCRIPTION OF THE DRAWINGS

The features of the present disclosure will be apparent from the following Detailed Description, taken in connection with the accompanying drawings, in which:

FIG. 1 is a block diagram illustrating the system of the present disclosure;

FIG. 2 depicts images of the needle tip enhancement process carried out by the system of the present disclosure;

FIG. 3 is a diagram illustrating the overall architecture of the deep CNN-LSTM network for needle tip localization, in accordance with the present disclosure;

FIG. 4 depict images illustrating needle tip enhancement and localization results produced by the systems and methods of the present disclosure, in three frames from one ultrasound sequence, obtained with in-plane insertion of a 22G needle in chicken tissue;

FIG. 5 is a diagram illustrating hardware and software components capable of implementing the computer vision systems and methods of the present disclosure; and

FIG. 6 is a diagram illustrating implementation of the systems and methods of the present disclosure in connection with an ultrasound scanner.

### DETAILED DESCRIPTION

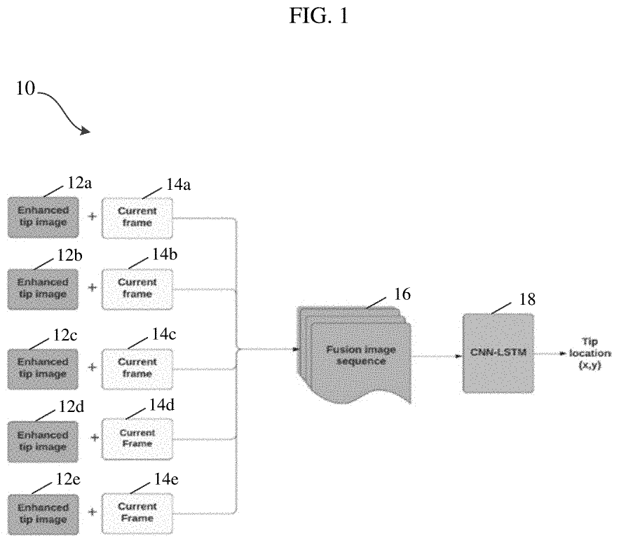

The present disclosure relates to computer vision systems and methods for time-aware needle tip localization in two-dimensional (2D) ultrasound images, as described in detail below in connection with FIGS. 1-6.

FIG. 1 is a block diagram illustrating the system of the present disclosure, indicated generally at 10. The system 10 processes a plurality of enhanced tip images 12a-12e and a plurality of current ultrasound frames 14a-14e to produce a fusion image sequence 16. The fusion image sequence is then processed by a convolutional neural network with long-short term memory (CNN-LSTM) 18 in order to identify the location of the tip of the needle. More particularly, the input to the neural network 18 includes a consecutive sequence of five fused images derived from enhanced tip images 12a-12e and the corresponding B-mode images 14a-14e.

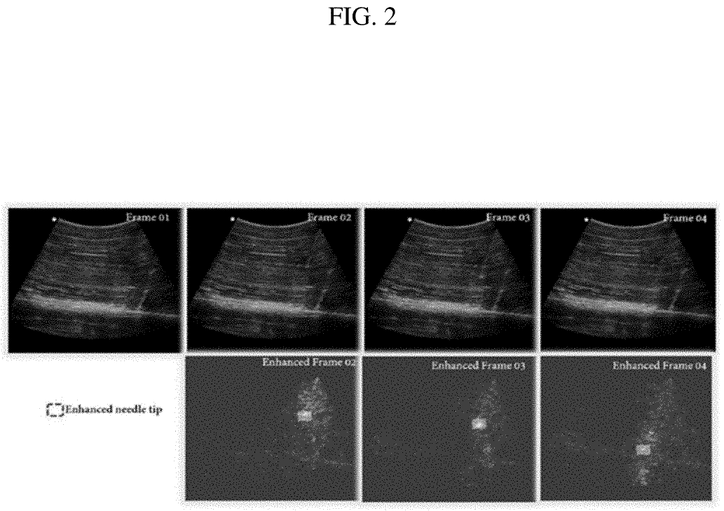

FIG. 2 depicts images of the needle tip enhancement process carried out by the system of the present disclosure. The needle tip enhancement process can be applied to four consecutive ultrasound frames, from data collected with in-plane insertion of a 17G needle in porcine tissue. Of course, other numbers and types of frames can be used, if desired. Row 1 of FIG. 2 depicts original B-mode ultrasound frames, US(x, y, t), and row 2 depicts needle tip enhanced images, USE(x, y). Notice that without the enhancement step, the needle tip is not easy to visualize with the naked eye.

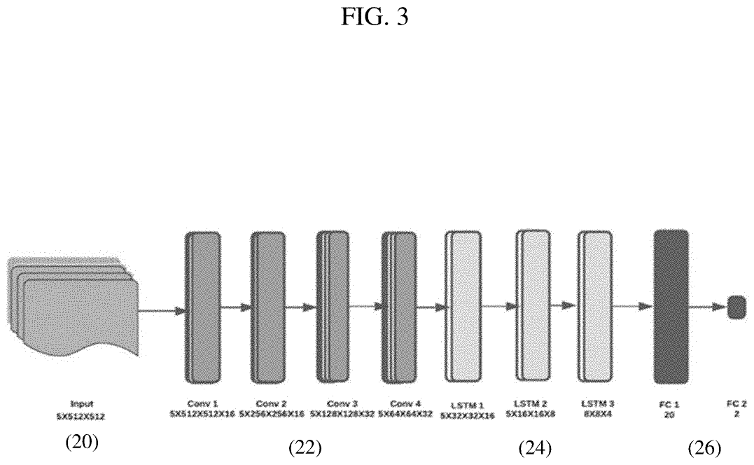

FIG. 3 is a diagram illustrating the overall architecture of the deep CNN-LSTM network for needle tip localization, in accordance with the present disclosure. L-R: Input data from five fused images 20 (enhanced tip image+corresponding B-mode image) are processed by four time-distributed convolutional layers 22. These are followed by convolutional LSTM layers 24 which model temporal dynamics associated with needle tip motion from the prior extracted activation maps, and lastly, two fully connected layers 26, whose final output is the tip location (x, y).

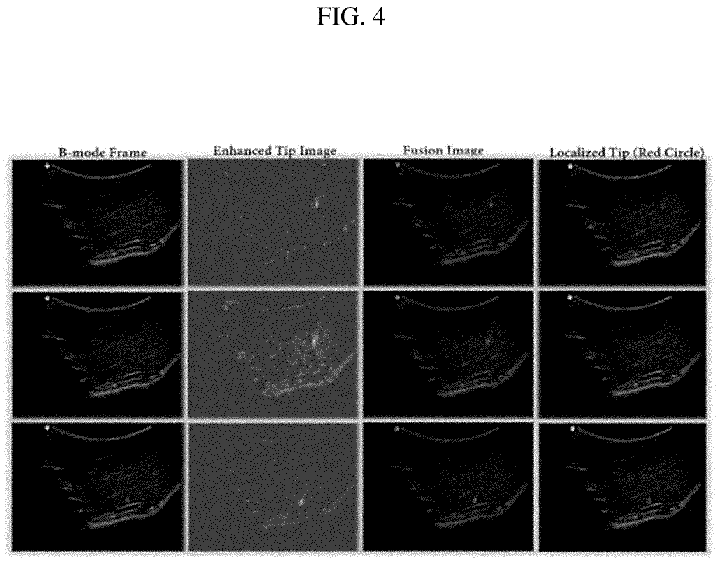

FIG. 4 depict images illustrating needle tip enhancement and localization results produced by the systems and methods of the present disclosure, in three frames from one ultrasound sequence, obtained with in-plane insertion of a 22G needle in chicken tissue. Column 1 shows the original B-mode ultrasound frames. Note that the needle tip is difficult to observe and shaft information is unavailable. Column 2 shows the needle tip enhanced image US_{E}(x, y) obtained using the method described in “Needle tip feature extraction from ultrasound frame sequences” section. Here, the tip appears as a characteristic high intensity in the image. Column 3 shows the fused image, derived from US_{E}(x, y) and the corresponding B-mode image. A consecutive sequence of five fused images is input to the CNN-LSTM network. Column 4 shows the tip localization result obtained from the CNN-LSTM model described in “Needle tip localization” section.

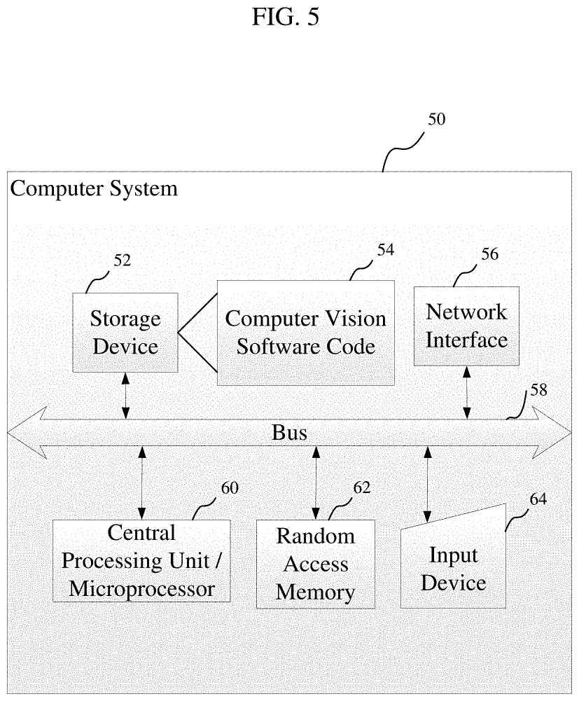

FIG. 5 is a diagram showing a hardware and software components of a computer system 50 on which the system of the present disclosure can be implemented. The computer system 50 can include a storage device 52, computer vision software code 54, a network interface 56, a communications bus 58, a central processing unit (CPU) (microprocessor) 60, a random access memory (RAM) 62, and one or more input devices 64, such as a keyboard, mouse, etc. The computer system 50 could also include a display (e.g., liquid crystal display (LCD), cathode ray tube (CRT), etc.). The storage device 52 could comprise any suitable, computer-readable storage medium such as disk, non-volatile memory (e.g., read-only memory (ROM), eraseable programmable ROM (EPROM), electrically-eraseable programmable ROM (EEPROM), flash memory, field-programmable gate array (FPGA), etc.). The computer system 50 could be a networked computer system, a personal computer, a server, a smart phone, tablet computer etc. It is noted that the computer system 50 need not be a networked server, and indeed, could be a stand-alone computer system.

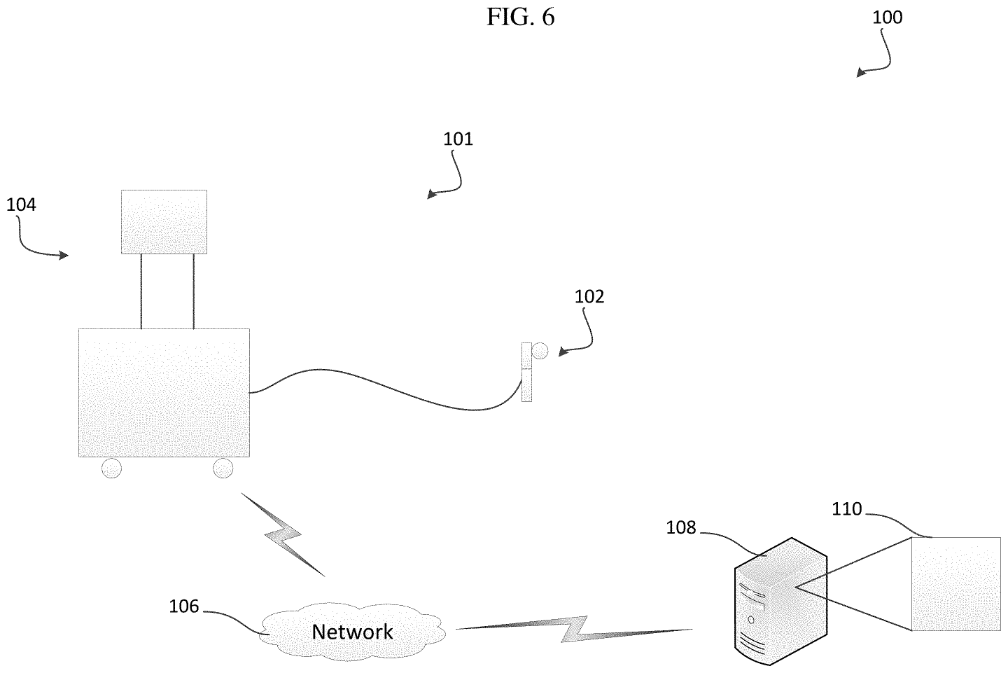

FIG. 6 is a drawing illustrating a system 100 of the present disclosure. The system can include an ultrasound device 101 including an ultrasound receiver 102 and an ultrasound base 104. The ultrasound receiver 102 is a device which can be used by a medical professional for placement on a patient to generate ultrasonic waves and receive a signal for generation of an ultrasonic video/image at the ultrasound base 104. The ultrasound device 101 can transmit data of the ultrasound video over the Internet 106 to a computer system 108 for localizing a medical device in an ultrasound video via a computer vision processing engine 110. It should be noted that the processing engine 110 can be located at the ultrasound base 104 obviating the need for the ultrasound device 101 to send data over the Internet 106 to a remote computer system.

The computer vision systems and methods discussed herein can be utilized in connection with hand-held 2D US probes during in-plane and out-of-plane needle insertion. The system splits the problem of motion-based needle localization into two parts: (a) motion detection in each frame and (b) spatial-temporal feature extraction. As illustrated in FIG. 1, the system first extracts needle tip features caused by otherwise imperceptible scene changes arising from needle motion in the 2D ultrasound image. This is achieved by logical subtraction of the current frame (foreground) from the previous frame, which acts as a dynamic reference (background). The system achieves an enhanced needle frame without requiring a priori information about the needle trajectory, and then fuses the enhanced tip images and the corresponding B-mode images and feed multiple consecutive fused images to a novel CNN-LSTM frame-work which localizes the needle tip in the last frame of the sequence. The needle tip feature extraction and enhancement processes that could be utilized with the present systems and methods include, but are not limited to, those processes disclosed in U.S. Patent Application Publication No. 2019/037829 to Mwikirize, et al. and International (PCT) Application No. PCT/US19/46364 to Mwikirize, et al., the entire disclosures of which are expressly incorporated herein by reference.

The systems and methods of the present disclosure were tested using 2D B-mode US data collected using two imaging systems: SonixGPS (Analogic Corporation, Peabody, Mass., USA) with a hand-held C5-2/60 curvilinear probe, at 30 frames-per-second (fps) and 2D hand-held wireless US (Clarius C3, Clarius Mobile Health Corporation, Burnaby, BC, Canada) at 24 fps. Three needle types were used: a 17G SonixGPS vascular access needle (Analogic Corporation, Peabody, Mass., USA), a 17G Tuohy epidural needle (Arrow International, Reading, Pa., USA), and a 22G spinal Quincke-type needle (Becton, Dickinson and Company, Franklin Lakes, N.J., USA). The needles were inserted in freshly excised bovine, porcine, and chicken tissue, with the chicken tissue overlaid on a lumbosacral spine phantom, in-plane (25° to 60°) and out-of-plane up to a depth of 70 mm. For experiments conducted with the SonixGPS needle, tip localization data was collected from the electromagnetic (EM) tracking system (Ascension Technology Corporation, Shelburne, Vt., USA). The data were collected by a clinician who introduced motion seen in clinical situations, via large probe pressure while con-currently rotating the transducer. 80 video sequences were collected (45 in-plane, 35 out-of-plane: 40 with SonixGPS system and 40 with Clarius C3 system), with each video sequence having more than 300 frames. The experiment particulars are shown in Table 1, below. Data for training and validation were extracted from 42 sequences. Test experiments were conducted on 600 frames extracted from 30 left out sequences. The test data were chosen to focus on sequences with large motion artifacts.

TABLE 1
Experimental details for 2D US data collection

| | | | Pixel |
|---|---|---|---|
| Imaging | Needle type, dimensions, | # of | size |
| system | insertion profile, tissue | videos | (mm) |
| Sonix | 17G SonixGPS (1.5 mm, 70 mm), IP, bovine | 5 | 0.17 |
| GPS | 17G Tuohy (1.5 mm, 90 mm), IP, bovine | 5 | 0.17 |
| | 22G BD (0.7 mm, 90 mm), IP, bovine | 5 | 0.17 |
| | 22G BD (0.7 mm, 90 mm), OP, bovine | 5 | 0.17 |
| | 17G SonixGPS (1.5 mm, 70 mm), OP, porcine | 5 | 0.17 |
| | 17G Tuohy (1.5 mm, 90 mm), IP, porcine | 5 | 0.17 |
| | 17G SonixGPS (1.5 mm, 70 mm), IP, chicken | 5 | 0.17 |
| | 22G BD (0.7 mm, 90 mm), OP, chicken | 5 | 0.17 |
| Clarius | 17G SonixGPS (1.5 mm, 70 mm), IP, bovine | 15 | 0.24 |
| C3 | 17G Tuohy (1.5 mm, 90 mm), IP, bovine | 15 | 0.24 |
| | 22G BD (0.7 mm, 90 mm), OP, porcine | 5 | 0.24 |
| | 22G BD (0.7 mm, 90 mm), OP, chicken | 5 | 0.24 |

IP in-plane insertion,
OP out-of-plane insertion

A temporal sequence of ultrasound frames was considered, with each frame denoted by the spatiotemporal function US(x, y, t), where t represents the time index and (x, y) are the spatial indexes. It is desirable to broadly categorize the pixels in each frame as either a foreground (needle tip) or back-ground (tissue). To achieve this, a dynamic background subtraction model can be utilized. To enhance the needle tip in frame US(x, y, t), we consider US(x, y, t−1), as a background, and perform the operation:

US_{E}(x,y)=US(x,y,y)∧US(x,y,t−1)^{c} (1)

Here, ∧ represents the bitwise AND logical operation, and (1) calculates the conjunction of pixels in the current frame and the logical complement of the preceding frame. Therefore, the output, US_{E}(x, y), contains only pixels that are in the present frame and not in the previous frame. Equation 1 is remarkably efficient at extracting the needle tip since it considers any spatiotemporal intensity variation between consecutive frames. To further enhance the needle tip, the output of Equation 1 is passed through a median filter with a 7×7 kernel. FIG. 2 illustrates a typical output of this enhancement approach on four consecutive frames (yielding three consecutive enhanced frames). The process of needle tip enhancement is almost cost-free and takes 0.0016 s on a 512×512 frame. Certainly, there could be other motion artifacts picked up by Equation 1, and therefore, the learning-based approach described next is important, to accurately localize the needle tip from US_{E}(x, y).

Enhanced tip images US_{E}(x, y) are derived from consecutive frames in a B-mode ultrasound sequence. It is expected that the tip feature in US_{E}(x, y) will exhibit a high intensity. Nevertheless, there are often motion artifacts or high-intensity artifacts arising from anatomy, which could be equally significant in the enhanced image. For this reason, we cannot rely on the highest intensity in US_{E}(x, y) to be the needle tip. To accurately localize the needle tip, we feed a plurality of fused images, comprising a combination of the tip enhanced images and the corresponding B-mode images, to a CNN-LSTM network, which associates spatial needle tip features in each enhanced frame, with the temporal information across the frame sequence.

As noted above (and also with reference to Table 2, below), FIG. 3 illustrates an architecture for a new deep neural network for needle tip localization, which combines convolutional and recurrent layers. The convolutional layers extract abstract representations of the input image data in feature maps. The recurrent layers implemented as LSTM layers pass previous hidden states to the next step of the sequence. The overall network holds information on previously seen data and uses it to make decisions.

TABLE 2
Architecture of the CNN-LSTM network

| N | Layer name | Output dimensions | Kernel | Filter number |
|---|---|---|---|---|
| 0 | Input | 5 × 512 × 512 × 1 | — | — |
| 1 | Conv 1 | 5 × 512 × 512 × 16 | 3 | 16 |
| 2 | Maxpool | 5 × 256 × 256 × 16 | 2 | — |
| 3 | Conv 2 | 5 × 256 × 256 × 16 | 3 | 16 |
| 4 | Maxpool | 5 × 128 × 128 × 16 | 2 | — |
| 5 | Conv 3 | 5 × 128 × 128 × 16 | 3 | 32 |
| 6 | Maxpool | 5 × 64 × 64 × 32 | 2 | — |
| 7 | Conv 4 | 5 × 64 × 64 × 32 | 3 | 2 |
| 8 | Maxpool | 5 × 32 × 32 × 32 | 2 | — |
| 9 | LSTM 1 | 5 × 32 × 32 × 16 | 3 | 16 |
| 10 | Maxpool | 5 × 16 × 16 × 16 | 2 | — |
| 11 | LSTM 2 | 5 × 16 × 16 × 8 | 3 | 8 |
| 12 | Maxpool | 5 × 8 × 8 × 8 | 2 | — |
| 13 | LSTM 3 | 8 × 8 × 4 | 3 | 4 |
| 14 | Maxpool | 4 × 4 × 4 | 7 | — |
| 15 | FC 1 | 20 | — | — |
| 16 | FC 2 | 2 | — | — |

The input to the network consists of a sequence of five fused images, with each image consisting of the enhanced tip image+the corresponding B-mode image. Using the fusion strategy instead of using only the enhanced tip image input is important because in case the needle tip does not move within the five-frame consecutive sequence, tip information is still available in the input, derived from the original B-mode frame (if there is no tip motion, US_{E}(x, y) is ideally all zeros, and does not contain any tip information). The frame number of 5 has been empirically determined based on optimizing computational efficiency of the network, while considering typical ultrasound frame rate and needle insertion speed for the data. Each input image is resized to 512×512. The input data feeds a series of four convolutional layers, which apply the respectively defined convolutional operations to each temporal slice in the input. The size of feature maps varies in different convolutional layers varies as shown in Table 1. All convolutional layers employ rectified linear units (ReLUs) activations, whose nonlinear function is defined as σ(x)=max(0, x). Each convolutional layer is followed by a max pooling layer, which also applies the max pooling operation to each temporal slice in the input at that stage. The convolutional max pool sequence is then followed by three convolutional LSTM layers, whose output size mirrors the input temporal sequence. Like the convolutional layers, the convolutional LSTM layers are interspersed with max pool layers. The last LSTM layer is followed by another max pooling layer, and two fully connected layers of size 20 and 2, respectively, since the desired model output is the tip position (x, y).

For data collected with the SonixGPS system, we derive ground-truth needle tip locations (x, y) in each frame using the inbuilt electromagnetic tracking system, cross-checked by an expert interventional radiologist with more than 25 years of experience. For data collected with the Clarius C3 system (which does not have a tracking solution), the ground-truth tip locations are determined via manual labeling by the expert radiologist. The labels are rescaled to be in the range [0, 1]. Following our desired output, we train our network as a regression CNN-LSTM, using Adam optimizer and mean squared error (MSE) loss. We trained and evaluated the model using Tensorflow in Google Colab, powered by the 12 GB Tesla K80 GPU parallel computing platform. The needle tip enhancement method was implemented in MATLAB 2019b on a 3.6 GHz Intel® Core™ i7 16 GB CPU Windows PC.

The performance of the systems and methods of the present disclosure was evaluated by comparing the automatically localized tip with the ground truth obtained from the electromagnetic tracking system for data collected with the SonixGPS needle. For data collected with needles without tracking capability (Tuohy and BD needles), the ground truth was determined by an expert sonographer. Tip localization accuracy was determined from the Euclidean distance between the corresponding measurements.

FIG. 4 illustrates the needle tip localization results for three consecutive frames, for in-plane needle insertion. These results show that the needle tip is accurately localized, even when it is not easily discernible with the naked eye. The proposed method performs well in the presence of high-intensity artifacts in the rest of the ultrasound image. Meanwhile, the proposed method is not sensitive to the type and size of the needle used in the experiments.

Two metrics can be used to compare the performance of the pro-posed method with existing state-of-the-art methods and variants of the current approach: tip localization error and total processing time. This comparison is shown in Table 3, below.

TABLE 3
Comparison of performance of the present system with
statement-of-the-art methods and alternative implementations.

| | | Processing | Error |
|---|---|---|---|
| | Error | time | below |
| Method | (mm) | (s) | 2 mm (%) |
| Proposed method | 0.52 ± 0.06 | 0.064 | 94 |
| Proposed method with raw input | 5.92 ± 1.5 | 0.062 | 4 |
| Alternative method 1 | 0.79 ± 0.15 | 0.092 | 76 |
| Alternative method 2 | 0.74 ± 0.08 | 0.021 | 81 |

On test data of 600 frames extracted from 30 ultra-sound sequences, the systems and methods of the present disclosure achieved a tip localization error of 0.52±0.06 mm and an overall computation time of 0.064 s (0.0016 s for frame enhancement and 0.062 s for model inference). Here, we consider the computational cost for enhancing one frame since we used a sliding window approach with a frame overlap of four frames in our model. We trained a similar CNN-LSTM model with input of raw B-mode ultrasound frames (without the tip enhancement step), while keeping the network's architecture and training detail constant. The resulting model performed poorly, with a tip localization error of 5.92±1.5 mm. This was not unexpected: without enhancement, the needle tip feature is often not distinct. Thus, the model could not learn the associated features, and this led to poor performance.

Next, the needle tip is enhanced, using a similar approach to the one described in this disclosure, and the resulting image is fed to a network derived from the YOLO architecture for needle tip detection. For a fair comparison, localization errors above 2 mm (24% compared to 6% of the test data with the proposed method) were not considered. This model achieves a localization error of 0.79±0.15 mm which is higher than that of the proposed method. A one-tailed paired t test shows that the difference between the localization errors from the proposed method and prior methods is statistically significant (p<0.005).

We also compared the present systems/methods with other approaches, where the needle tip is also first enhanced, and then fed to a model cascade of classifier and a location regressor. This approach achieves a localization error of 0.74±0.08 mm (81% of the test data below 2 mm error), with statistically significant under-performance compared to the proposed method (p<0.005). It is not hard to under-stand why the proposed method is superior to the other approaches: the current approach takes as input an enhanced sequence of needle tip images and hence learns spatiotemporal information related to both structure and motion behavior of the needle tip. The previous methods, on the other hand, take in one frame at a time and do not learn any temporal information. This makes them prone to artifacts that may look like the needle tip, especially when they are outside the needle trajectory.

Having thus described the system and method in detail, it is to be understood that the foregoing description is not intended to limit the spirit or scope thereof. It will be understood that the embodiments of the present disclosure described herein are merely exemplary and that a person skilled in the art may make any variations and modification without departing from the spirit and scope of the disclosure. All such variations and modifications, including those discussed above, are intended to be included within the scope of the disclosure. What is desired to be protected by Letters Patent is set forth in the following claims.

What is claimed is:

1. A computer vision method for time-aware needle tip localization in two-dimensional (2D) ultrasound images, comprising the steps of:
receiving at a processor a plurality of images of a needle tip;
receiving at the processor a plurality of B-mode frame corresponding to the plurality of images;
processing the plurality of images and the plurality of B-mode frames to generate a fused image sequence;
processing the fused image sequence using a time-aware neural network to detect a tip location; and
identifying the tip location.

2. The method of claim 1, wherein the plurality of images of the needle tip comprise a plurality of enhanced images of the needle tip.

3. The method of claim 1, wherein the time-aware neural network comprises a unified convolutional neural network (CNN) and long-short term memory (LSTM) recurrent neural network.

4. The method of claim 3, wherein the CNN comprises four time-distributed convolutional layers.

5. The method of claim 4, wherein the LSTM recurrent neural network comprises a plurality of convolutional LSTM layers which model temporal dynamics associated with needle tip motion.

6. The method of claim 5, further comprising a plurality of fully connected layers processing output of the plurality of convolutional LSTM layers to identify the tip location.

7. The method of claim 1, wherein the fused image sequence comprises a consecutive sequence of fused images.

8. The method of claim 1, wherein the plurality of images comprise a plurality of ultrasound images.

9. The method of claim 5, wherein the plurality of B-mode frames comprise a plurality of B-mode ultrasound frames.

10. The method of claim 1, wherein the processor is part of an ultrasound device.

11. A computer vision system for time-aware needle tip localization in two-dimensional (2D) ultrasound images, comprising:
a memory storing a plurality of images of a needle tip and a plurality of B-mode frame corresponding to the plurality of images; and
a processor in communication with the memory, the processor:
processing the plurality of images and the plurality of B-mode frames to generate a fused image sequence;
processing the fused image sequence using a time-aware neural network to detect a tip location; and
identifying the tip location.

12. The system of claim 11, wherein the plurality of images of the needle tip comprise a plurality of enhanced images of the needle tip.

13. The system of claim 11, wherein the time-aware neural network comprises a unified convolutional neural network (CNN) and long-short term memory (LSTM) recurrent neural network.

14. The system of claim 13, wherein the CNN comprises four time-distributed convolutional layers.

15. The system of claim 14, wherein the LSTM recurrent neural network comprises a plurality of convolutional LSTM layers which model temporal dynamics associated with needle tip motion.

16. The system of claim 15, further comprising a plurality of fully connected layers processing output of the plurality of convolutional LSTM layers to identify the tip location.

17. The system of claim 11, wherein the fused image sequence comprises a consecutive sequence of fused images.

18. The system of claim 11, wherein the plurality of images comprise a plurality of ultrasound images.

19. The system of claim 15, wherein the plurality of B-mode frames comprise a plurality of B-mode ultrasound frames.

20. The system of claim 11, wherein the processor is part of an ultrasound device.
